(19) United States
(12) Patent Application Publication
(10) Pub. No.: US 2023/0210867 A1
(43) Pub. Date: Jul. 6, 2023

(54) TREATMENT OF ADJUSTMENT DISORDERS

(71) Applicant: VISTAGEN THERAPEUTICS, INC., South San Francisco, CA (US)

(72) Inventors: Shawn K. SINGH, South San Francisco, CA (US); Mark A. SMITH, South San Francisco, CA (US)

(21) Appl. No.: 17/998,823

(22) PCT Filed: May 18, 2021
(86) PCT No.: PCT/US2021/032920

§ 371 Date: Nov. 15, 2022

Related U.S. Application Data
(60) Provisional application No. 63/026,441, filed on May 18, 2020.

(51) Int. Cl. A61K 31/568; A61P 25/22; A61K 9/00

(52) U.S. Cl. CPC A61K 31/568; A61P 25/22; A61K 9/0043

(57) ABSTRACT

This invention relates to methods of treating adjustment disorders, particularly adjustment disorder with anxiety (AjDA), in an individual include administering a therapeutically effective amount of an androsta-4,16-dien-3-ol steroid to the individual. In some embodiments of the methods, a therapeutically effective amount of an androsta-4,16-dien-3-ol steroid is administered to a patient, and in some embodiments of the compositions, a therapeutically effective amount of an androsta-4,16-dien-3-ol is included in a pharmaceutical composition for the treatment of AjDA.

### CROSS-REFERENCE TO RELATED APPLICATIONS

This application claims priority to U.S. Application 63/026,441 filed May 18, 2020, which is incorporated herein by reference.

### FIELD OF THE INVENTION

This invention relates to methods and compositions for treating adjustment disorders, particularly adjustment disorder with anxiety (AjDA).

### BACKGROUND TO THE INVENTION

The unsettling and unprecedented uncertainty associated with the COVID-19 pandemic has caused tremendous stress and anxiety worldwide, especially related to health, healthcare, safety and economic and personal financial matters. It is not only the recent onset of extraordinary fear, uncertainty and anxiety surrounding the actual impact or potential threat of physical illness from the coronavirus itself, but also the impact of mass quarantine and social separation which can bring about confusion, irritability, insomnia, nervousness, worry, jitteriness and the like. In addition, the ominous new and uncertain future economic consequences of the COVID-19 pandemic and related personal financial strains are often overwhelming for many.

Adjustment disorders have been known to occur when sudden events introduce new stress into one's life—such as the loss of a job, a loved one's death, unexpected changes in relationships, etc. See, https://www.healthline.com/health/adjustment-disorder, (accessed May 18, 2020) for a discussion of adjustment disorders and their symptoms. People worldwide are now experiencing adjustment disorders due to the difficulty coping with the sudden stress of the COVID-19 pandemic in their life. All ages, including children and adults, may suffer from adjustment disorders when a stressful event occurs. Symptoms, both mental and physical, typically occur immediately after or during the event and typically last about six months but may last longer if the stress continues. One particular adjustment disorder is adjustment disorder with anxiety (AjDA). People suffering with AjDA may feel overwhelmed, anxious and worried and may also have problems with concentration and memory.

As a result of the COVID-19 pandemic, in recent months, there has been a substantial (>30%) increase in the prescription of anti-anxiety and antidepressant drugs in the U.S. Although prevalence rates fall between 11% and 18% in people who attend primary care, in many respects, current treatment alternatives for AjDA fall short of patient needs due to troubling side effects and safety concerns and/or slow onset of even limited effect. New generation treatment options with rapid-onset activity but without troubling side effects and safety concerns are needed.

There is therefore a need for an effective treatment for adjustment disorder with anxiety (AjDA), including a treatment that can be effectively administered when a sufferer from AjDA has experienced or continues to experience a stressful life event.

### SUMMARY OF THE INVENTION

The invention relates to methods of treating an adjustment disorder, such as adjustment disorder with anxiety (AjDA), in an individual comprising the step of administering a therapeutically effective amount of an androsta-4,16-dien-3-ol neuroactive steroid to the individual in need thereof. The invention also relates to the use of a therapeutically effective amount of an androsta-4,16-dien-3-ol steroid to treat an adjustment disorder in an individual in need thereof. The androsta-4,16-dien-3-ol steroid is an androsta-4,16-dien-3-ol, for example, [3β]-androsta-4,16-dien-3-ol and [3a]-androsta-4,16-dien-3-ol. The androsta-4,16-dien-3-ol steroid is administered by nasal administration, such as vomeronasal administration and administration to the nasal chemosensory mucosa. The androsta-4,16-dien-3-ol steroid is administered in a pharmaceutical composition comprising the androsta-4,16-dien-3-ol steroid and a pharmaceutically acceptable carrier. In methods of the invention the pharmaceutical composition is an aqueous solution of the androsta-4,16-dien-3-ol steroid.

### DESCRIPTION OF THE INVENTION

When nasally administered to human subjects, androsta-4,16-dien-3-ol steroids, and particularly [3β]-androsta-4,16-dien-3-ol, are effective to treat adjustment disorders (AjD), particularly AjDA, in human individuals. These compounds are believed to specifically bind to chemosensory receptors of certain nasal neuroepithelial cells and it is believed that this binding generates a series of neurophysiological responses that aid in the alleviation of symptoms and in the treatment of social anxiety disorder in humans. One or both enantiomers of androsta-4,16-dien-3-ol steroids may be administered to a human subject to treat social anxiety disorder. The β enantiomer, [3β]-androsta-4,16-dien-3-ol (also termed “androsta-4,16-dien-3β-ol”), appears to be more potent and is odorless. The α enantiomer ([3α]-androsta-4,16-dien-3-ol (also termed “androsta-4,16-dien-3α-ol”)) has a pleasant odor, and may be administered to patients and included in compositions with the β enantiomer to impart a pleasant odor as well as for its pharmacological effects.

The chemical structures of the two enantiomers of androsta-4,16-dien-3-ol are illustrated below, with [3β]-androsta-4,16-dien-3-ol shown as Formula (I):

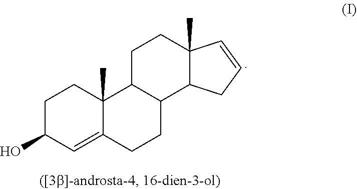 [SMILES: C[C@@]12C=CCC1C1CCC3=C[C@@H](O)CC[C@]3(C)C1CC2]

[3α]-androsta-4,16-dien-3-ol shown as Formula II:

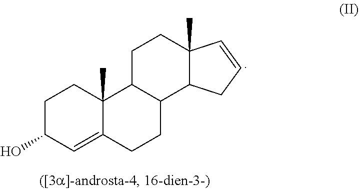 [SMILES: C[C@@]12C=CCC1C1CCC3=C[C@H](O)CC[C@]3(C)C1CC2]

Pharmaceutical composition of an androsta-4,16-dien-3-ol compounds, such as [3β]-androsta-4,16-dien-3-ol and methods of making them are described in U.S. Pat. No. 8,309,539 B2, which is incorporated herein by reference. The pharmaceutical composition may be an ointment, a powder, a liquid, or an aerosol. The methods also include preparing a pharmaceutical composition containing [3α]-androsta-4,16-dien-3-ol, or may include preparing a pharmaceutical composition containing both [3β]-androsta-4,16-dien-3-ol and [3α]-androsta-4,16-dien-3-ol, in which the androsta-4,16-dien-3-ol compound or compounds may be dissolved in a pharmaceutically acceptable carrier, an ointment, a powder, a liquid, or an aerosol.

This invention relates to methods of treating adjustment disorders, particularly adjustment disorder with anxiety (AjDA), in an individual include administering a therapeutically effective quantity of an androsta-4,16-dien-3-ol steroid to the individual. In some embodiments of the methods, a therapeutically effective amount of an androsta-4,16-dien-3-ol steroid is administered to a patient, and in some embodiments of the compositions, a therapeutically effective amount of an androsta-4,16-dien-3-ol is included in a pharmaceutical composition for the treatment of AjDA. An “effective amount” of androsta-4,16-dien-3β-ol is the amount that, when administered to the vomeronasal organ of a person suffering from AjDA, is sufficient to achieve treatment of the AjDA. The effective quantity is a quantity that, if administered systemically, would be ineffective to achieve treatment of the AjDA but that is effective when administered to the vomeronasal organ (VNO) and the nasal chemosensory mucosa.

Therapeutically effective amounts may be, for example, between about 100 picograms and about 100 micrograms, or between about 1 nanogram and about 10 micrograms, or between about 10 nanograms and about 1 microgram of an androsta-4,16-dien-3-ol. The androsta-4,16-dien-3-ol compound is preferably administered to the nasal passages and the vomeronasal organ of the individual. The individual is preferably a human subject. In embodiments, the androsta-4,16-dien-3-ol steroid is [3β]-androsta-4,16-dien-3-ol. In embodiments of the methods, the individual is a woman. In further embodiments, the androsta-4,16-dien-3-ol steroid is [3α]-androsta-4,16-dien-3-ol. In some embodiments of the methods, both [3β]-androsta-4,16-dien-3-ol and [3α]-androsta-4,16-dien-3-ol are administered to a patient, and in some embodiments of the compositions, both [3β]-androsta-4,16-dien-3-ol and [3α]-androsta-4,16-dien-3-ol are included in a pharmaceutical composition for the treatment of AjDA.

In one aspect, the invention provides a method for the treatment of AjDA, comprising administering an effective amount of an androsta-4,16-dien-3-ol, such as androsta-4,16-dien-3β-ol, to the vomeronasal organ of a person in need of such treatment. In embodiments of the methods, the compound is preferably [3β]-androsta-4,16-dien-3-ol. In further embodiments, the methods may comprise the step of administering to the nasal passages and the vomeronasal organ (VNO) of an individual both [3β]-androsta-4,16-dien-3-ol and [3α]-androsta-4,16-dien-3-ol. In embodiments, the step of administering may comprise administering to the nasal passages and the vomeronasal organ of the individual a unit dosage of an androsta-4,16-dien-3-ol. A unit dosage of androsta-4,16-dien-3-ol, such as a unit dosage of [3β]-androsta-4,16-dien-3-ol, may be up to about 100 micrograms of the compound or compounds. In embodiments of the invention, the quantity of androsta-4,16-dien-3-ol, such as [3β]-androsta-4,16-dien-3-ol, that is administered to the patient is between about 100 picograms and about 100 micrograms. In other embodiments, the amount of the androsta-4,16-dien-3-ol compound or compounds, such as [3β]-androsta-4,16-dien-3-ol, that is administered is between about 1 nanogram and about 10 micrograms, or between about 10 nanograms and about 1 microgram.

Pharmaceutical compositions administered in a method of the invention may have about 100 micrograms of androsta-4,16-dien-3-ol steroid in a pharmaceutically acceptable carrier or may have between about 100 picograms and about 100 micrograms. In embodiments, the pharmaceutical composition includes between about 1 nanogram and about 10 micrograms of androsta-4,16-dien-3-ol, or between about 10 nanograms and about 1 microgram of androsta-4,16-dien-3-ol. The pharmaceutically acceptable carrier may be combined with the androsta-4,16-dien-3-ol to provide an ointment, a powder, a liquid, or an aerosol. Thus, the pharmaceutical compositions may include [3β]-androsta-4,16-dien-3-ol, [3α]-androsta-4,16-dien-3-ol or both.

The pharmaceutical formulations used herein contain one or more pharmaceutically acceptable carriers (also termed excipients or vehicles) suited for the particular type of formulation, i.e. vapor, liquid, gel, ointment or the like. The vehicles are comprised of naturally occurring or synthetic compounds or materials that do not adversely affect androsta-4,16-dien-3β-ol or other components of the formulation. Suitable carriers for use herein include water, silicone, waxes, petroleum jelly, propylene glycol, liposomes, and a variety of other materials. The pharmaceutical formulations administered are typically contained within drug delivery systems known in the art which provide a specific, predetermined agent release profile, i.e.: single dose or multidose metered delivery device. Such systems can include for example metered spray actuators, aerosols and nasal inhalers.

In a method of the invention, the androsta-4,16-dien-3-ol neuroactive steroid may administered in a pharmaceutical composition comprising the androsta-4,16-dien-3-ol neuroactive steroid and a pharmaceutically acceptable carrier. Such pharmaceutical compositions are described in, for example, U.S. Pat. Nos. 5,883,087; 6,057,439; 8,309,539; and 8,722,562, which are incorporated here by reference. The pharmaceutical composition may be an ointment, a powder, a liquid, or an aerosol. The pharmaceutical composition may be an aqueous solution of the androsta-4,16-dien-3-ol steroid, particularly when administered using nasal administration.

In such a pharmaceutical composition, the androsta-4,16-dien-3-ol neuroactive steroid, for example, androsta-4,16-dien-3β-ol (formula (I), also known as PH84B) may be formulated in water and delivered intranasally in spray form to treat an adjustment disorder, such as adjustment disorder with anxiety (AjDA). Due to the hydrophobic properties of the steroid, dissolution in water was performed with the aid of co-solvents propylene glycol (0.5% to 5% w/v), ethanol (0.1% to 5% w/v) and Tween 80-R (0.01% to 2% w/v) and water (q.s. to 100%). Benzalkonium chloride (0.01% w/v) was used as a preservative. A commercially available, metered-dose, spray pump may be used for to deliver a dose of the androsta-4,16-dien-3-ol steroid, e.g. androsta-4,16-dien-3β-ol, intranasally. After the spray pump was primed, each activation may deliver a 25-250 μL, a 75-200 μL, a 50-150 μL, a 75-125 μL, a 80-120 μL, or a 100 μL spray of fine microdroplets and may deliver about 0.5-5 μg, about 0.75-2.55 μg, about 1-2 μg, about 1.25-1.75 μg, about 1.5 μg or about 1.6 μg of an androsta-4,16-dien-3-ol steroid, e.g. androsta-4,16-dien-3β-ol, intranasally. For example, each spray may deliver about 1.6 μg of an androsta-4,16-dien-3-ol steroid per 100 μL spray. The effective quantity delivered to the nasal mucosa in the area of the medial and dorsal nasal septum is typically achieved by spraying intranasally with the spray nozzle directed toward the VNO opening and positioned about 1 centimeter from the anterior and lower nasal septum.

Adjustment disorder (AjD) refers to a maladaptive emotional and/or behavioral response to an identifiable stressor occurring within three (3) months of the onset of the stressor as evidenced by one (1) or both of the following:

(1) marked distress that is out of proportion to the severity or intensity of the stressor, taking into account socially or culturally expected reactions; and/or

(2) significant impairment or interference with a person's social, occupational or other important areas of daily functioning.

Unlike post-traumatic stress disorder (PTSD) or acute stress disorder, which have clear criteria for what constitutes a traumatic event, AjD criteria do not specify any requirements for what may be regarded as a stressor giving rise to AjD. However, research has identified that identifiable stressor events may include both traumatic events, such as exposure to actual or threatened death or illness of a loved one, oneself or others (e.g., stress experienced by healthcare workers and first responders related to the COVID-19 pandemic), as well as non-traumatic stressful events, such as interpersonal conflict, unemployment, and financial difficulties (e.g., unemployment or fear of loss of employment or healthcare benefits, or loss of business due to government-imposed business shut-down, shelter-in-place and/or social distancing orders related to COVID-19). Importantly, the stress reaction can be so great in AjD that patients attempt suicide. Indeed, patients diagnosed with AjD have a 12-fold increased rate of suicide compared to controls.

The COVID-19 pandemic has caused tremendous and unprecedented uncertainty, stress and anxiety worldwide, especially related to health and healthcare, safety and economic and personal financial matters. It is not only the recent onset of unsettling and extraordinary fear and anxiety surrounding the actual impact or potential threat of illness from the coronavirus itself, but also the mass quarantine and social separation which can bring about confusion, irritability, insomnia, nervousness, worry, jitteriness and the like. In addition, COVID-19 pandemic-induced unemployment, economic loss and related financial strains are tremendous. As a result, in recent months, there has been a substantial (>30%) increase in the prescription of anti-anxiety and antidepressant drugs in the U.S.

According to DSM-5, there are six (6) subtypes of AjD that feature symptoms of the following: (i) depressed mood; (ii) anxiety; (iii) mixed anxiety and depressed mood; (iv) disturbance of conduct; (v) mixed disturbance of emotions and conduct; and (vi) unspecified. AjD is a distinct and different condition than generalized anxiety disorder, social anxiety disorder and specific phobias.

Because PH94B has been shown to be safe in all clinical studies to date, and has demonstrated significant efficacy in treating symptoms of social anxiety disorder (SAD), and because its antidepressant properties are as yet unknown, the present Phase 2A study will be focused on adjustment disorder with anxiety (AjDA).

Current treatments for AjD include both psychosocial and pharmacologic measures. Psychosocial treatments include exposure therapy and cognitive behavioral therapy (CBT). Pharmacological measures vary widely and include antidepressants (such as selective serotonin reuptake inhibitors (SSRIs) and serotonin norepinephrine reuptake inhibitors (SNRIs)), anxiolytics (such as benzodiazepines), buspirone and natural products (such as cannabidiol). Current treatments are not effective in all subjects and induce known adverse effects that may lead to discontinuation of treatment.

### Example 1: Aqueous Formulation

An exemplary aqueous pharmaceutical formulation for use as a neuroactive nasal spray in a method of the invention is described in Table 1. In this example the androsta-4,16-dien-3-ol steroid is [3β]-androsta-4,16-dien-3-ol (Formula (I) also known as PH94B).

TABLE 1
Nasal Spray Pharmaceutical Composition

| Component | Function | Concentration | |
|---|---|---|---|
| [3β]-androsta-4,16-dien-3-ol (PH94B) | API | 16 | μg/ml |
| Phosphate Buffer Solution pH 7 | Buffering | 8.6% | v/v |
| | Agent | | |
| Tween 80 | Surfactant | 0.3% | w/v |
| Propylene glycol | Solvent | 2.0% | v/v |
| Ethyl alcohol (ethanol) | Solvent | 3.0% | v/v |
| Benzalkonium chloride 50% | Preservative | 0.01% | w/v |

| | | |
|---|---|---|
| Sterile Water for Injection | Solvent | q.s. to volume |

API = active pharmaceutical ingredient;
q.s. = quantity sufficient

### Example 2: Clinical Trial Protocol for Assessment of AjDA Symptoms Associated with Identifiable Stressors Related to the COVID-19 Pandemic

A clinical trial is designed and conducted to evaluate the efficacy, safety and tolerability of daily administration of PH94B neuroactive nasal spray (Example 1) as a treatment of Adjustment Disorder with Anxiety (AjDA) symptoms in adults, associated with identifiable stressors related to the COVID-19 pandemic. Subject are confirmed with current diagnoses of this condition as defined in the Diagnostic and Statistical Manual of Mental Disorders, 5th Edition (DSM-5).

The PH94B study drug is delivered intranasally with the nozzle of the applicator positioned in the nasal vestibule and directed toward the midline (medial part or nasal septum). In order to administer study medication, the actuator (pusher) should be depressed.

Prior to the use of a new study medication bottle, the subject is instructed to shake the bottle five times and to prime the bottle by depressing the actuator five times. After the activation of the device, it will deliver 1.6 μg of the medication with each spray. Subjects are instructed to shake the bottle three times prior to each dose, but no further priming should be done.

Subjects are instructed to administer one (1) 1.6 μg spray of study medication into each nostril (right and left nasal passages), for two (2) total sprays per dose (for a total of 3.2 μg). Subjects are instructed to point the spray nozzle toward the nasal septum (nasal division) while using the spray.

During the clinical trial, PH94B is self-administered by the subject four (4) times per day. So, for example, if the subject is anxious, fearful, worried, or nervous excessively about something directly or indirectly related to the current COVID-19 pandemic, such as fear of contracting or unknowingly spreading the disease by going out in public or to work, potential unemployment, current financial issues, interpersonal conflicts or concerns due to social isolation, etc., the patient will self-administer PH94B four (4) times per day.

Men and women will be treated with the same dose of PH94B: 3.2 μg. Each nasal spray delivers 100 μL containing 1.6 μg PH94B. The nasal retention volume of an adult of either sex is 200-250 μL. Therefore, the spray volume administered to each nostril (100 μL) will be below the nasal retention volume and will be retained in the nasal passages. One (1) dose of study drug is defined as one (1) spray (100 μL) administered to each nostril, two (2) sprays total per dose, for a total dose of 3.2 μg of PH94B. Patients are instructed to wait a minimum of 1-2 hours before self-administering PH94B again.

The total duration of drug administration is four (4) weeks+/−two (2) days.

The primary endpoint for this Phase 2A study is the change in the Hamilton Anxiety Scale (HAM-A) total score from Baseline to the end of four (4) weeks of treatment with PH94B compared to placebo.

Secondary endpoints are selected from the following.

1. Individual subject improvement as assessed by the Sheehan Disability Scale (SDS) at the end of week 4 of treatment with PH94B compared to placebo.

2. Individual subject improvement as assessed by the Clinical Global Impression-Improvement rating (CGI-I). Values for comparison will be the proportion of “responders” in each group, defined as subjects receiving scores of 1 (Very much improved) or 2 (Much improved) during the fourth week of treatment with PH94B compared to placebo.

3. Individual subject improvement as assessed by the subject's self-assessment of improvement (PGI-C) at the end of week 4 of treatment with PH94B compared to placebo.

4. Individual subject improvement on the Adjustment Disorder New Module (ADNM) scale at the end of week 4 of treatment with PH94B compared to placebo.

Upon completion of the clinical trial, it is shown that one or more of the adult subjects with AjDA who participated experience a statistically significant reduction in anxiety levels as measured by the Hamilton Anxiety Scale (HAM-A). It is also shown that one or more subjects have a statistically significant improvement in the secondary endpoints.

The claimed invention is:

1. A method of treating an adjustment disorder in an individual comprising the step of administering a therapeutically effective amount of an androsta-4,16-dien-3-ol steroid to the individual in need thereof.

2. A method of claim 1, wherein the adjustment disorder is adjustment disorder with anxiety (AjDA).

3. A method of claim 1 or claim 2, wherein the androsta-4,16-dien-3-ol steroid is an androsta-4,16-dien-3-ol.

4. A method of any one of claims 1-3, wherein the androsta-4,16-dien-3-ol steroid is [313]-androsta-4,16-dien-3-ol.

5. A method of any one of claims 1-3, wherein the androsta-4,16-dien-3-ol steroid is [3a]-androsta-4,16-dien-3-ol.

6. A method of any one of claims 1-5, wherein the androsta-4,16-dien-3-ol steroid is administered by nasal administration or administration to the nasal chemosensory mucosa.

7. A method of any one of claim 6, wherein the nasal administration is vomeronasal administration or administration to the nasal chemosensory mucosa.

8. A method of any one of claims 1-7, wherein the androsta-4,16-dien-3-ol steroid is administered in a pharmaceutical composition comprising the androsta-4,16-dien-3-ol steroid and a pharmaceutically acceptable carrier.

9. A method of claim 8, wherein the pharmaceutical composition is an ointment, a powder, a liquid, or an aerosol.

10. A method of claim 8 or claim 9, wherein the pharmaceutical composition is an aqueous solution of the androsta-4,16-dien-3-ol steroid.

11. A method of claim 10, wherein the androsta-4,16-dien-3-ol steroid is [313]-androsta-4,16-dien-3-ol.

12. A method of claim 10, wherein the androsta-4,16-dien-3-ol steroid is [3a]-androsta-4,16-dien-3-ol.

13. Use of a therapeutically effective amount of an androsta-4,16-dien-3-ol steroid to treat an adjustment disorder in an individual in need thereof.

14. The use of claim 13, wherein the adjustment disorder is adjustment disorder with anxiety (AjDA).

15. The use of claim 13 or claim 14, wherein the androsta-4,16-dien-3-ol steroid is an androsta-4,16-dien-3-ol.

16. The use of any one of claims 13-15, wherein the androsta-4,16-dien-3-ol steroid is [313]-androsta-4,16-dien-3-ol.

17. The use of any one of claims 13-15, wherein the androsta-4,16-dien-3-ol steroid is [3a]-androsta-4,16-dien-3-ol.
